SEQUENCE LISTING (19 sequences)

SEQ ID NO: 1 - DNA, 60 bp, Artificial sequence
  Synthetic sequence
agcccctgcccaccgcacactgcgctgccccagacccactgtgcgtgtgacagcggctga

SEQ ID NO: 2 - DNA, 35 bp, Artificial sequence
  Synthetic sequence
acgactcactataggagcccctgcccaccgcacac

SEQ ID NO: 3 - DNA, 21 bp, Artificial sequence
  Synthetic sequence
tcagccgctgtcacacgcaca

SEQ ID NO: 4 - RNA, 110 nt, Homo sapiens
acccggcagugccuccaggcgcagggcagccccugcccaccgcacacugcgcugccccagacccacugugcgugugacagcggcugaucugugccugggcagcgcgaccc

SEQ ID NO: 5 - RNA, 112 nt, Homo sapiens
ccaccccgguccugcucccgccccagcagcacacugugguuuguacggcacuguggccacguccaaaccacacugugguguuagagcgagggugggggaggcaccgccgagg

SEQ ID NO: 6 - RNA, 80 nt, Homo sapiens
gcguguguauauguguugcauguguguauauguguguauauauguacacauacacauacacgcaacacauauauacaugc

SEQ ID NO: 7 - RNA, 84 nt, Homo sapiens
gaugcacccagugggggagccaggaaguauugauguuucugccaguuuagcgucaacacuugcugguuuccucucuggagcauc

SEQ ID NO: 8 - RNA, 100 nt, Homo sapiens
ugugucucucucuguguccugccagugguuuuacccuaugguagguuacgucaugcuguucuaccacaggguagaaccacggacaggauaccggggcacc

SEQ ID NO: 9 - RNA, 86 nt, Homo sapiens
agaggacuccaaggaacauucaacgcugucggugaguuugggauuugaaaaaaccacugaccguugacuguaccuugggguccuua

SEQ ID NO: 10 - RNA, 83 nt, Homo sapiens
cugacuaugccuccccgcauccccuagggcauugguguaaagcuggagacccacugccccaggugcugcuggggguuguaguc

SEQ ID NO: 11 - RNA, 94 nt, Homo sapiens
aucacagacaccuccaagugugcagggcacuggugggggccggggcaggcccagcgaaagugcaggaccuggcacuuagucggaagugagggug

SEQ ID NO: 12 - RNA, 78 nt, Homo sapiens
uacugcccucggcuucuuuacagugcugccuuguugcauauggaucaagcagcauuguacagggcuaugaaggcauug

SEQ ID NO: 13 - RNA, 78 nt, Homo sapiens
ugcagccugggcgacaaaacgagacccugucuuuuuuuuuuucugagacagaggucucguucuguugcccaagcugga

SEQ ID NO: 14 - RNA, 80 nt, Homo sapiens
ugccccuggucugaguuccuggagccuggucugucacuggggaaguccagagcuccaaggcucagugcccaggggacgca

SEQ ID NO: 15 - RNA, 81 nt, Homo sapiens
gucaugugacugccugucugugccugcuguacaggugagcugauguucugcacagcaaguguagacaggcagacacaugac

SEQ ID NO: 16 - RNA, 90 nt, Homo sapiens
guuaccugguagugaguuagagaugcagagcccugggcuccucaucagcaaaccuacuggaucugcauuuuaauucacaugcaugguaau

SEQ ID NO: 17 - RNA, 67 nt, Homo sapiens
cugguccauuucccugccauucccuuggcuucaauuuacucccagggcuggcagugacaugggucaa

SEQ ID NO: 18 - RNA, 81 nt, Homo sapiens
cucucugcuuucagcuucuuuacaguguugccuuguggcauggaguucaagcagcauuguacagggcuaucaaagcacaga

SEQ ID NO: 19 - RNA, 87 nt, Homo sapiens
ugguuggguuuggauuguuguacuuuuuuuuuuguucguugcauuuuuaggaacaaaaaaaaaagcccaacccuucacaccacuuca

(19) United States
(12) Patent Application Publication
(10) Pub. No.: US 2023/0041228 A1
(43) Pub. Date: Feb. 9, 2023

(54) Small Molecule Inhibition of Micro-RNA-210 Reprograms an Oncogenic Hypoxic Circuit

(71) Applicant: University of Florida Research Foundation, Incorporated, Gainesville, FL (US)

(72) Inventor: Matthew D. Disney, Jupiter, FL (US)

(21) Appl. No.: 16/486,211

(22) PCT Filed: Feb. 16, 2018
(86) PCT No.: PCT/US18/18512

§ 371 Date: Aug. 15, 2019

Related U.S. Application Data
(60) Provisional application No. 62/460,201, filed on Feb. 17, 2017.

(51) Int. Cl. A61K 31/496; A61P 35/00

(52) U.S. Cl. CPC A61K 31/496; A61P 35/00

(57) ABSTRACT

Herein, we describe the identification of a small molecule named Targapremir-210 that binds to the Dicer site of the miR-210 hairpin precursor. This interaction inhibits production of the mature miRNA, de-represses glycerol-3-phosphate dehydrogenase 1-like enzyme (GPD1 L), a hypoxia-associated protein negatively regulated by miR-210, decreases HIF-1 a, and triggers apoptosis of triple negative breast cancer cells only under hypoxic conditions. Further, Targapremir-210 inhibits tumorigenesis in a mouse xenograft model of hypoxic triple negative breast cancer. We applied Chemical Cross-Linking and Isolation by Pull Down (Chem-CLIP) to study the cellular selectivity and the on- and off-targets of Targapremir-210. Targapremir-210 selectively recognizes the miR-210 precursor and can differentially recognize RNAs in cells that have the same target motif but have different expression levels, revealing this important feature for selectively drugging RNAs for the first time.

### CROSS-REFERENCE TO RELATED APPLICATION

This application claims the priority of U.S. provisional application Ser. No. 62/460,201, filed Feb. 17, 2017, the disclosure of which is incorporated herein by reference in its entirety.

### STATEMENT OF GOVERNMENT SUPPORT

This invention was made with government support under R01-GM097455 awarded by the National Institutes of Health. The government has certain rights in the invention.

### BACKGROUND

RNA plays pervasive and important roles in cellular biology and can contribute to disease pathology. Although 80% of DNA is transcribed into RNA, only 1.5% is translated into protein^{1,2}. Not surprisingly, non-coding RNAs contribute to disease, cementing RNA as an important therapeutic target. Much effort has been invested in the development of small molecules targeting protein, however, the identification of small molecule modulators of RNA has only been sparsely reported. Thus, one long-standing challenge in drug discovery and chemical biology is selectively drugging human RNAs with small molecules. To fill this void, we developed various synergistic approaches that enable the rational design of small molecules that target RNA from sequence. One foundational approach is Two-Dimensional Combinatorial Screening (2DCS), a library-versus-library screen that quickly defines the preferred RNA motifs for a given small molecule³. The data from 2DCS are compiled into a database that is mined against folded RNA structures within the human transcriptome to identify potentially druggable RNA targets from sequence via an approach named Inforna^{4,6}. MicroRNAs (miRNAs) are one class of RNA drug targets that are at the forefront of drug discovery efforts. These small, non-coding RNAs negatively regulate protein expression by targeting the 3′ untranslated regions (UTRs) of mRNAs, leading to their translational repression or cleavage⁶⁻⁸. Numerous miRNAs are associated with disease pathology⁹. For example, microRNA (miR)-210 is a central regulator of the hypoxic response that affects expression of hypoxia inducible factors (HIFs) in solid tumor masses¹⁰. MiR-210 represses levels of the glycerol-3-phosphate dehydrogenase 1-like (GPD1L) enzyme, contributing to suppression of prolyl hydroxylase (PHD) activity¹⁰⁻¹². Under normal physiological conditions, PHD hydroxylates prolines in hypoxia inducible factor 1-alpha (HIF-1α), leading to its degradation by the proteasome.¹³ When PHD activity is suppressed due to downregulation of GPD1L by miR-210, HIF-1α is not degraded by the proteasome¹⁰ and translocates to the nucleus where it forms a heterodimer with hypoxia-inducible factor 1-beta (HIF-1β); dimerization of HIF-1□ and HIF-1β activates transcriptional responses that contribute to cancer metastasis (FIG. 1A)¹⁴.

### SUMMARY

A hypoxic state is critical to the metastatic and invasive characteristics of cancer. Numerous pathways play critical roles in cancer maintenance, many of which include non-coding RNAs such as microRNA (miR)-210 that regulates hypoxia inducible factors (HIFs). Herein, we describe the identification of a small molecule named Targapremir-210 that binds to the Dicer site of the miR-210 hairpin precursor. This interaction inhibits production of the mature miRNA, de-represses glycerol-3-phosphate dehydrogenase 1-like enzyme (GPD1L), a hypoxia-associated protein negatively regulated by miR-210, decreases HIF-1α, and triggers apoptosis of triple negative breast cancer cells only under hypoxic conditions. Further, Targapremir-210 inhibits tumorigenesis in a mouse xenograft model of hypoxic triple negative breast cancer. Many factors govern molecular recognition of biological targets by small molecules. For protein, chemoproteomics and activity-based protein profiling are invaluable tools to study small molecule target engagement and selectivity in cells. Such approaches are lacking for RNA, leaving a void in the understanding of its druggability. We applied Chemical Cross-Linking and Isolation by Pull Down (Chem-CLIP) to study the cellular selectivity and the on- and off-targets of Targapremir-210.

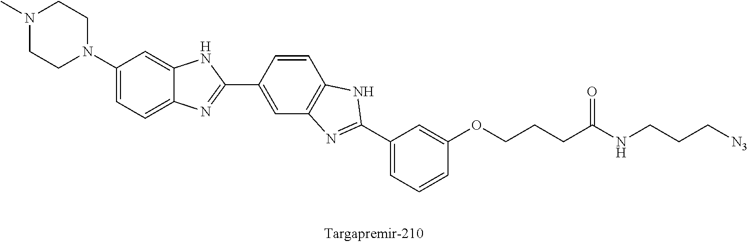 [SMILES: CN1CCN(c2ccc3nc(-c4ccc5[nH]c(-c6cccc(OCCCC(=O)NCCCN=[N+]=[N-])c6)nc5c4)[nH]c3c2)CC1]

Targapremir-210 selectively recognizes the miR-210 precursor and can differentially recognize RNAs in cells that have the same target motif but have different expression levels, revealing this important feature for selectively drugging RNAs for the first time. These studies show that small molecules can be rapidly designed to selectively target RNAs and affect cellular responses to environmental conditions, resulting in favorable benefits against cancer. Further, they help define rules for identifying druggable targets in the transcriptome.

In various embodiments, the invention provides a method of inhibiting biogenesis of microRNA miR-210 in a cell under hypoxic conditions, comprising contacting the cell with an effective amount or concentration of a compound of formula

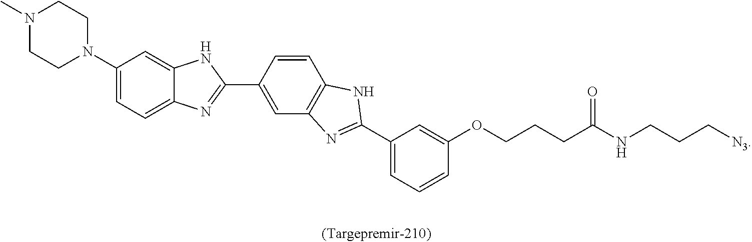 [SMILES: CN1CCN(c2ccc3nc(-c4ccc5[nH]c(-c6cccc(OCCCC(=O)NCCCN=[N+]=[N-])c6)nc5c4)[nH]c3c2)CC1]

In various embodiments of the invention, the Targapremir-210 selectively recognizes the miR-210 precursor and can differentially recognize RNAs in cells that have the same target motif but have different expression levels.

The invention further provides, in various embodiments, a method of target profiling a set of cells for expression of a miR-210 microRNA precursor, comprising administering an effective amount or concentration of Targapermir-210 to the set of cells, then screening the set of cells for expression or suppression of a phenotype associated with miR-210.

The invention further provides, in various embodiments, a method of impeding tumor proliferation in a mammal in vivo, comprising administering to the animal an effective dose of a compound of of formula

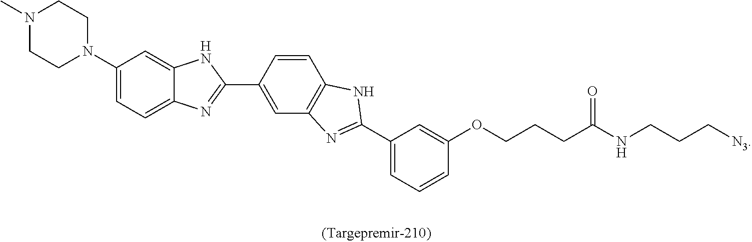 [SMILES: CN1CCN(c2ccc3nc(-c4ccc5[nH]c(-c6cccc(OCCCC(=O)NCCCN=[N+]=[N-])c6)nc5c4)[nH]c3c2)CC1]

For instance, the tumor can be breast cancer.

### BRIEF DESCRIPTION OF THE FIGURES

FIGS. 1A and 1B. Overview of miR-210 and Targapremir-210 activity. FIG. 1A) Schematic of miR-210's regulatory effect on GPD1L and HIF-1α in hypoxia, which contributes to the metastasis of cancer cells. GPD1L, glycerol-3-phosphate dehydrogenase 1-like enzyme; HIF-1α, hypoxia-inducible factor 1-alpha; HIF-1α, hypoxia-inducible factor 1-beta; PHD, prolyl hydroxylase; UB, ubiquitin; VHL, Von Hippel-Lindau tumor suppressor (E3 ubiquitin protein ligase). FIG. 1B) Schematic of an approach to inhibit miR-210 biogenesis with a designed small molecule, Targapremir-210.
FIG. 2. Targapremir-210 inhibits processing of pre-miR-210 by Dicer in vitro. Left, Representative gel image of the inhibition of pre-miR-210 processing by Dicer as a function of Targapremir-210 concentration. Nucleotides protected from Dicer cleavage are boxed and indicated in miR-210's secondary structure (Right, top). A “G ladder” was generated by digestion with RNase T1; OH ladder indicates a base hydrolysis ladder. Right, secondary structure of pre-miR-210 and quantification of protection from Dicer cleavage. Nucleotides protected from Dicer cleavage are boxed in the secondary structure.
FIGS. 3A, 3B, and 3C. Biological activity of Targapremir-210. FIG. 3A) Expression of various biomolecules in MDA-MB-231 cells under hypoxic conditions, showing increased levels of pri-, pre-, and mature miR-210 and HIF-1α and decreased levels of GPD1L. *, p<0.05 compared to normoxic expression, as determined by a two-tailed Student t-test. FIG. 3B) Effect of Targapremir-210 (200 nM) on levels of mature miR-210 in MDA-MB-231 cells cultured under hypoxic conditions, as determined by RT-qPCR. *, p<0.05 and **, p<0.01 compared to untreated, as determined by a two-tailed Student t-test. FIG. 3C) Effect of Targapremir-210 (200 nM) and an antagomir directed against miR-210 on pri- and pre-miR-210, HIF-1α, and GPD1L levels in MDA-MB-231 cells as determined RT-qPCR. The dashed line represents no change in RNA levels as compared to untreated MDA-MB-231 cells. *, p<0.05 compared to untreated, as determined by a two-tailed Student t-test.
FIGS. 4A and 4B. Effect of Targapremir-210 or anti-miR-210 antagomir on MDA-MB-231 cell survival in hypoxia or normoxia. FIG. 4A) Targapremir-210 (200 nM) and an antagomir directed against miR-210 (anti-miR-210; 50 nM) trigger apoptosis in MDA-MB-231 cells cultured under hypoxic conditions, as determined by Annexin V/PI staining and analysis by flow cytometry. Overexpression of pre-miR-210 ablated the ability of Targapremir-210 to trigger apoptosis. FIG. 4B) Targapremir-210 (200 nM) and anti-miR-210 (50 nM) do not trigger apoptosis in MDA-MB-231 cells cultured in normoxic conditions, as determined by Annexin V/PI staining and analysis by flow cytometry.
FIGS. 5A and 5B. Cellular selectivity of Targapremir-210 in MDA-MB-231 cells. FIG. 5A) Amongst miRNAs upregulated in MDA-MB-231 cells under hypoxic conditions, miR-210 is the only one significantly affected by Targapremir-210 (200 nM), with a similar signature as an antagomir (50 nM), as determined by RT-qPCR. The dashed line represents no change in RNA levels as compared to untreated MDA-MB-231 cells. FIG. 5B) Amongst miRNAs that contain motifs that bind Targapremir-210 with various affinities, only miR-210 expression levels are affected by the compound in MDA-MB-231 cells cultured under hypoxic conditions, as determined by RT-qPCR. The value indicated in parentheses after the miRNA identifier indicates its normalized expression level, as compared to miR-210, in untreated cells. ^{a} predicted affinityl¹⁵; *, p<0.05 as determined by a two-tailed Student t-test.
FIGS. 6A, 6B, and 6C. Evaluation of Targapremir-210 target engagement. FIG. 6A) Structures of small molecules used to study Targapremir-210 via for Chemical Cross-Linking and Isolation by Pull Down (Chem-CLIP). The control Chem-CLIP probe, Control-CA-Biotin, lacks an RNA-binding module. FIG. 6B) In vitro experiments show that the Targapremir-210-CA-Biotin Chem-CLIP probe selectively binds to the miR-210 hairpin precursor as compared to a CA-biotin probe lacking the RNA-binding module. FIG. 6C) Treatment of MDA-MB-231 cells with Targapremir-210-CA-Biotin affords ˜20-fold enrichment of pre-miR-210 in the pulled down fraction, indicating reaction with miR-210 in cells. Addition of Targapremir-210 inhibits reaction of Targapremir-210-CA-Biotin with miR-210, indicating on-target effects of the probe and the designed small molecule. *, p<0.05 as determined by a two-tailed Student t-test.
FIGS. 7A, 7B, and 7C. Targapremir-210 impedes tumor proliferation in vivo. FIG. 7A) Live bioluminescent imaging of NOD/SCID mice 3 weeks post-implantation of MDA-MB-231-GFP-Luc cells either pretreated in vitro with Targapremir-210 or miR-210 antagomir (top) or administered with a single injection of Targapremir-210 or miR-210 antagomir 24 h post-implantation (bottom). FIG. 7B) Resected tumor masses from NOD/SCID mice administered with a single injection of either Targapremir-210 or miR-210 antagomir 24 h post tumor implantation. FIG. 7C) Real-time qPCR of resected tumor masses demonstrating on-target effects of miR-210 inhibition by Targapremir-210. * indicates p<0.05 compared to untreated mice, as determined by a two-tailed Student t-test.

### DETAILED DESCRIPTION

Small molecule inhibition of miR-210 could perturb this complex hypoxic circuit, leading to various favorable benefits for the treatment and study of cancer. Herein, we report that a small molecule bis-benzimidazole identified by Inforna^{4,5}, Targapremir-210 (FIG. 1B), binds miR-210's Dicer processing site and modulates the miR-210 hypoxic circuit in triple negative breast cancer cells and a mouse xenograft model.

### Biological Activity of Targapremir-210

We first assessed the ability of Targapremir-210 to inhibit Dicer processing of the miR-210 hairpin precursor (pre-miR-210) in vitro. As shown in FIG. 2, Targapremir-210 inhibited pre-miR-210 processing at nanomolar concentrations, which correlated well with its binding affinity for the pre-miR-210 Dicer site, 5′ACU3′/3′UCA5′ (K_{d}˜200 nM¹⁵. Importantly, Targapremir-210 did not bind an RNA in which the Dicer site was ablated¹⁵. Targapremir-210 binds similarly to an in vitro transcribed pre-miR-210. Thus, Targapremir-210 bound avidly to pre-miR-210 and binding was sufficient to inhibit its processing in vitro.

To establish a cellular model to assess inhibition of miR-210 processing, we compared the expression profiles of various hypoxia-associated biomolecules in MDA-MB-231 triple negative breast cancer (TNBC) cells. As shown in FIG. 3A, mature miR-210 levels were increased by 15-fold under hypoxic conditions, with similar effects observed for primary (pri-) and pre-miR-210. Concomitant changes were observed for GPD1L mRNA expression (10-fold reduction) and HIF-1α mRNA expression (10-fold increase) (FIG. 3A), as expected¹⁰. Increased levels of HIF-1α and miR-210 have been previously established in other breast cancer cell lines and in tissues^{13,16,17}. Taken together, the MDA-MB-231 cell line serves as an appropriate model to study modulation of miR-210 by small molecules under hypoxic conditions.

In agreement with our in vitro studies demonstrating inhibition of pre-miR-210 Dicer processing by Targapremir-210 (FIG. 2), the compound decreased mature miR-210 levels in MDA-MB-231 cells cultured under hypoxic conditions, with an IC₅₀ of ˜200 nM (FIG. 3B). Concomitant increases were observed in pri- (˜3-fold) and pre-miR-210 (˜2.6-fold) levels upon treatment with 200 nM Targapremir-210 (FIG. 3C), indicating that the compound's mode of action is inhibition of Dicer processing and not transcriptional silencing. An antagomir directed against miR-210, (anti-miR-210; 50 nM) decreased levels of the mature form of the miRNA but does not affect pri- and pre-miR-210 levels, as expected (FIG. 3C).

### Cellular Responses of Targapremir-210 in TNBC Cells

Given that Targapremir-210 decreases levels of mature miR-210, we studied the downstream cellular responses to Targapremir-210 treatment on MDA-MB-231 cells. As expected, HIF-1α mRNA levels in hypoxic MDA-MB-231 cells were reduced by ˜75% following treatment with 200 nM of Targapremir-210 while GPD1L levels were increased by ˜4-fold, similar to the effects observed upon treatment with anti-miR-210 (FIG. 3C). Thus, addition of the small molecule reverted the MDA-MB-231 genotypic expression levels towards that of a normoxic state, consistent with Targapremir-210 inhibiting miRNA biogenesis. Under hypoxic conditions, upregulation of HIF-1α prevents cells from entering apoptosis^{18,19}. Since Targapremir-210 treatment reduced HIF-1α levels, we studied whether the compound could trigger apoptosis in MDA-MB-231 cells cultured under hypoxic conditions. Indeed, the compound induced apoptosis, as assessed by Annexin V/propidium iodide (PI) staining (FIG. 4A). Furthermore, this response was selective for the hypoxic environment as Targapremir-210 did not induce apoptosis in MDA-MB-231 cells cultured in normoxia (FIG. 4B). Notably, overexpression of pre-miR-210 allowed cells to escape apoptosis in the presence of the compound, suggesting that increased levels of the pre-miRNA overloaded the dosage of the compound (FIG. 4A). Thus, the small molecule has desirable genotypic and phenotypic effects by inhibiting miR-210 biogenesis.

### Cellular Selectivity: Hypoxia-Associated and Off-Target miRNAs

We next studied the cellular selectivity of Targapremir-210 and compared it with an antagomir directed against miR-210 by RT-qPCR. In particular, we analyzed the effect of compound treatment on a panel of hypoxia-associated miRNAs (n=28)²⁰. Interestingly, both Targapremir-210 (200 nM) and anti-miR-210 (50 nM) affected only levels of mature miR-210, indicating that the compound is at least as selective as the oligonucleotide-based antagomir (FIG. 5A).

To further test the selectivity of Taragpremir-210, we compared the effect of compound and miR-210 antagomir treatment using a microarray analysis of ˜2500 miRNAs. Both the small molecule and antagomir had similar miRNA specificity profiles. These studies confirm the selectivity of Targapremir-210 is similar to that of a gene-specific antagomir on a transcriptome-wide level.

### Cellular Selectivity: RNA Isoforms

There is extensive knowledge of protein isoform-specific targeting^{21,22.} Likewise, RNAs that have motifs that are recognized by the same small molecule represent, as we now term, RNA isoforms. One important feature of Inforna is its ability to identify privileged RNA motifs for a given small molecule and assign a Fitness Score to each (100 being the most fit) as a measure of selectivity^{4,5}. Indeed, a 2DCS selection of Targapremir-210 identified other small molecule-binding RNA motifs. Notably, these motifs have lower Fitness Scores and weaker affinities than the Dicer binding site in pre-miR-210¹⁵. We queried a database of secondary structural elements present in miRNA hairpin precursors²³ and identified miRNAs containing the pre-miR-210 Dicer site motif and other motifs that can bind to Targapremir-210, albeit with lower affinity (Table 1). Amongst all miRNAs with predicted compound binding sites, Targapremir-210 only affected levels of miR-210 (FIG. 5B).

### Direct Target Engagement of Pre-miR-210 by Targepremir-210

To determine if Targapremir-210 directly engages the miR-210 hairpin precursor, we employed an approach developed in our laboratory named Chemical Cross-Linking and Isolation by Pull-Down (Chem-CLIP)²⁴. Briefly, a derivative of Targapremir-210 was synthesized that contains chlorambucil (CA) cross-linking and biotin purification modules (Targapremir-210-CA-Biotin; FIG. 6A).

Targapremir-210 drives binding to pre-miR-210, bringing chlorambucil into close proximity of the RNA such that they react to form a covalent cross-link. Biotin is then used to capture the cross-linked RNA on streptavidin beads. In vitro validation showed that the Targapremir-210 Chem-CLIP probe reacted selectively with a miR-210 hairpin precursor, as compared to the control Chem-CLIP compound without the RNA-binding module, which showed little to no reaction with the miR-210 precursor (FIG. 6B).

In MDA-MB-231 cells grown under hypoxic conditions, Targapremir-210-CA-Biotin reacted with the miR-210 hairpin precursor, increasing the abundance of the target by greater than 20-fold in the purified fraction, when using primers that amplify only pre-miR-210 (FIG. 6C). A competitive Chem-CLIP (C-Chem-CLIP) experiment was completed to assess if cross-linking of the target is due to non-specific reactivity of the reactive (CA) module. In C-Chem-CLIP, cells are treated with the Chem-CLIP probe and increasing concentrations of the unreactive, parent compound. A target that binds selectively to the parent compound will be depleted as a function of concentration. A C-Chem-CLIP experiment completed in MDA-MB-231 cells showed that 200 nM of Targapremir-210 was required to inhibit ˜50% of the reaction of Targapremir-210-CA-Biotin with the miR-210 hairpin precursor (FIG. 6C), which correlates well with the measured K_{d} of the compound with the pre-miR-210 Dicer site.

### Direct Target Engagement of Highly Abundant Transcripts by Targapremir-210

To analyze the transcriptome-wide targets that Targapremir-210 engages in cells, we profiled the levels of 92 highly abundant transcripts in the pulled down fraction by Chem-CLIP. The RNAs analyzed included ribosomal (r)RNAs, small (s)RNAs, transfer (t)RNAs, and messenger (m)RNAs that span the diverse population of the transcriptome²⁵. Amongst the 92 abundant transcripts, only six were present to a greater extent in the pulled-down fraction, with the largest relative pull-down equal to ˜2-fold; levels of mature miR-210 were enriched by greater than 7-fold. To determine if pull-down of the six enriched RNAs was due to non-selective binding caused by the chlorambucil and/or biotin modules, we completed a Chem-CLIP experiment with the Control-CA-Biotin compound (FIG. 6A) that lacks the RNA-binding module. Interestingly, for three of the RNAs, there is no significant difference between the amount of RNA pulled down by Targapremir-210-CA-Biotin and Control-CA-Biotin.

### Direct Target Engagement of Hypoxia-Associated miRNAs by Targapremir-210

We also assessed the abundance of hypoxia-associated miRNAs in the pulled down fraction. Indeed, miR-210 had the greatest abundance amongst the pulled down miRNAs by an order of magnitude. Enrichment was only observed for three of 28 of the miRNAs studied (miR-181a, miR-205, and miR-206), with a highest relative fold pull-down of ˜2.8-fold. To determine if enrichment might be caused by non-selective binding of the CA and biotin modules to the hypoxia-associated or miRNAs, a C-Chem-CLIP experiment was completed. Compared to the C-Chem-CLIP results for miR-210, the abundance of other hypoxia-associated miRNAs did not change as a function of Targapremir-210 concentration, suggesting pull-down of these off-targets may be in part due to non-selective effects of the CA or biotin moieties.

### Direct Target Engagement of RNA Isoforms by Targapremir-210

We next determined if the 15 miRNAs that have an RNA isoform that binds Targapremir-210 are bound in MDA-MB-231 cells. By comparing the ability of Targapremir-210 to knock down mature levels of these miRNAs by RT-qPCR and to bind them by Chem-CLIP, we can assess: (i) if small molecules can differentially bind to RNAs that have the same RNA motif based on their expression level; (ii) if binding to non-functional sites (not Drosha and Dicer processing sites) has a biological effect; and (iii) other factors that can affect ligand occupancy of on- and off-targets.

Chem-CLIP studies revealed that only four of the miRNAs are bound by Targapremir-210 in MDA-MB-231 cells, miR-497, miR-1273c, miR-3174, and miR-107. Yet, the compound has no statistically significant effect on any of their expression levels. Interestingly, miR-497 contains the exact same motif as miR-210's Dicer site but the motif is located outside Dicer or Drosha processing sites. Further, miR-497 levels are 10-fold lower than miR-210, which is reflective of its decreased enrichment as compared to miR-210. A Targapremir-210 binding site is present in the Dicer or Drosha processing sites of miR-324, miR-3174, and miR-4446. All three miRNAs are lower abundance as compared to miR-210 (at least 2-fold) and their predicted binding sites are >10-fold less avid than miR-210's Dicer site.

The cellular occupancy of miR-1273c by Targapremir-210 is an interesting case, as the same motif (5′CCU/3′GAA) that binds the compound is present in four miRNAs that were not enriched in Chem-CLIP studies, miR-648, miR-103a, miR-4682, and miR-3120. MiR-648 and miR-3120 are expressed at much lower levels than miR-1273c (>10-fold), which could explain why they are not occupied in cellulis. In contrast, miR-103a abundance is similar to miR-1273c 's while miR-4682 is 2-fold higher. Careful inspection of miR-1273c's secondary structure provides potential insight into its greater occupancy by Targapremir-210 as compared to miR-103a and miR-4682: its secondary structure contains three CA internal loop motifs that could be bound by Targapremir-210.

Collectively, these data suggest that: (i) simple binding is not sufficient to elicit a biological effect; rather, binding must occur to a functional (Dicer or Drosha processing) site; (ii) the compound must bind avidly to a processing site; and (iii) the degree of target occupancy depends on the abundance of the RNA and the affinity of the small molecule.

The Chem-CLIP approach also has implications for determining RNA secondary structure. Chemical modification reagents such as dimethyl sulfate (DMS)²⁶⁻²⁸ and Selective 2′-Hydroxyl Acylation and Primer Extension (SHAPE) reagents²⁹ have³⁰ provided invaluable tools to constrain RNA structure predictions from sequence. Likewise, the observation that small molecule recognize specific RNA motifs in cells could provide invaluable constraints to refine cellular RNA structures.

### Targapremir-210 as an RNA-Binding Module

Interestingly, Targaprimir-96⁴, a dimer that selectively targets pri-miR-96, displays Targapremir-210 as one of its RNA-binding modules. Thus, we tested if Targaprimir-96 affects miR-210 levels and if Targapremir-210 affects miR-96 levels under hypoxic conditions. Importantly, we found that Targaprimir-96 only affects miR-96 and that Targapremir-210 only affects miR-210. Thus, molecular recognition in cells is governed by the RNA-binding module in the context of the entire ligand. Such properties have been shown previously^{31,32}.

### Pharmacological Testing of Targapremir-210

Previously, compounds within the bis-benzimidazole class have been used as topoisomerase inhibitors³³. Topoisomerases catalyze the breakage and rejoining of the DNA backbone, and topoisomerase inhibitors are known to be efficient inducers of apoptosis³⁴. Thus, we tested if Targapremir-210 inhibited topoisomerase enzymatic activity in vitro. At 200 nM, the active concentration used in our TNBC cell studies, Targapremir-210 did not inhibit topoisomerase activity, suggesting that it triggers apoptosis in TN BC cells via on-target inhibition of miR-210 biogenesis, in agreement with RT-qPCR data that showed increases in pri-and pre-miR-210 levels (FIG. 3C) and the loss of compound activity upon overexpression of pre-miR-210 (FIG. 4B).

Other bis-benzimidazoles are currently used as fluorescent stains for DNA, such as Hoechst 33342. Hoechst 33342 is structurally similar to Targapremir-210 except the former phenol is para substituted as opposed to meta substituted. Therefore, we measured the affinity of Hoechst 33342 for pre-miR-210. No saturable binding was observed when up to 5000 nM pre-miR-210 was added, indicating that it binds much less avidly than Targapremir-210. Similarly, Hoechst 33342 (200 nM; approximate IC₅₀ of Targapremir-210) has no statistically significant effect on mature miR-210 levels in hypoxic MDA-MB-231 cells.

### Targapremir-210 Decreases Tumor Burden in a TNBC Mouse Model

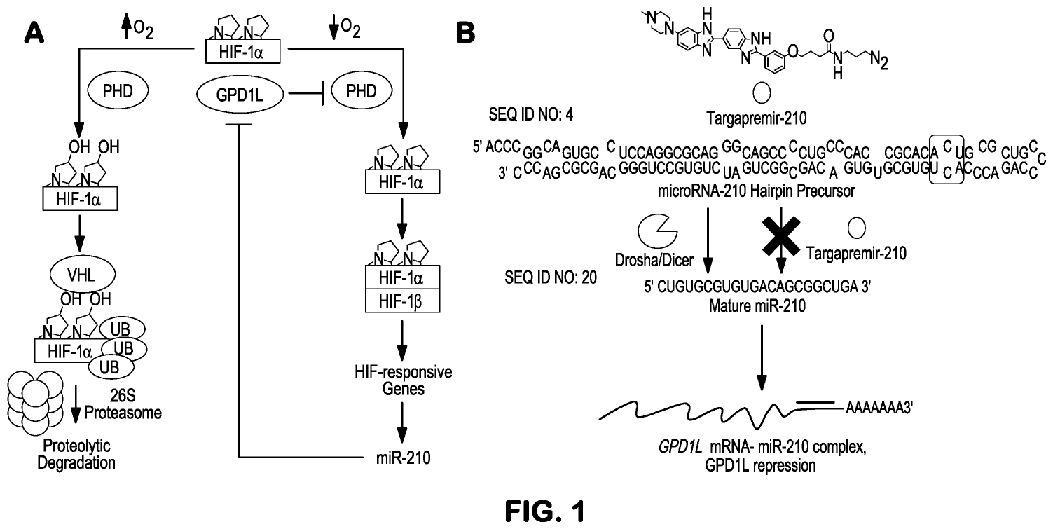

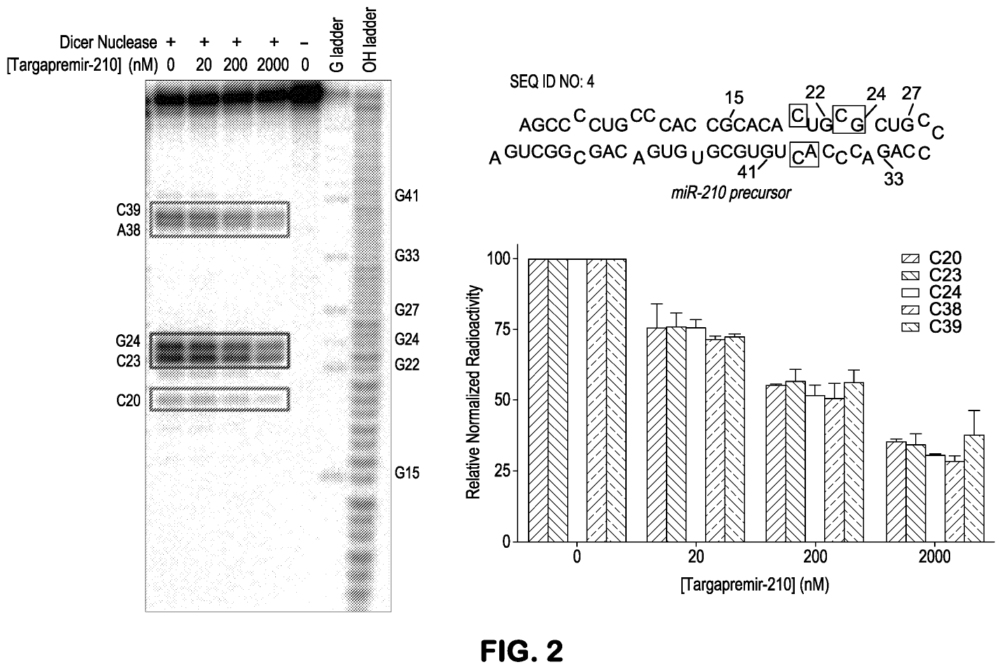

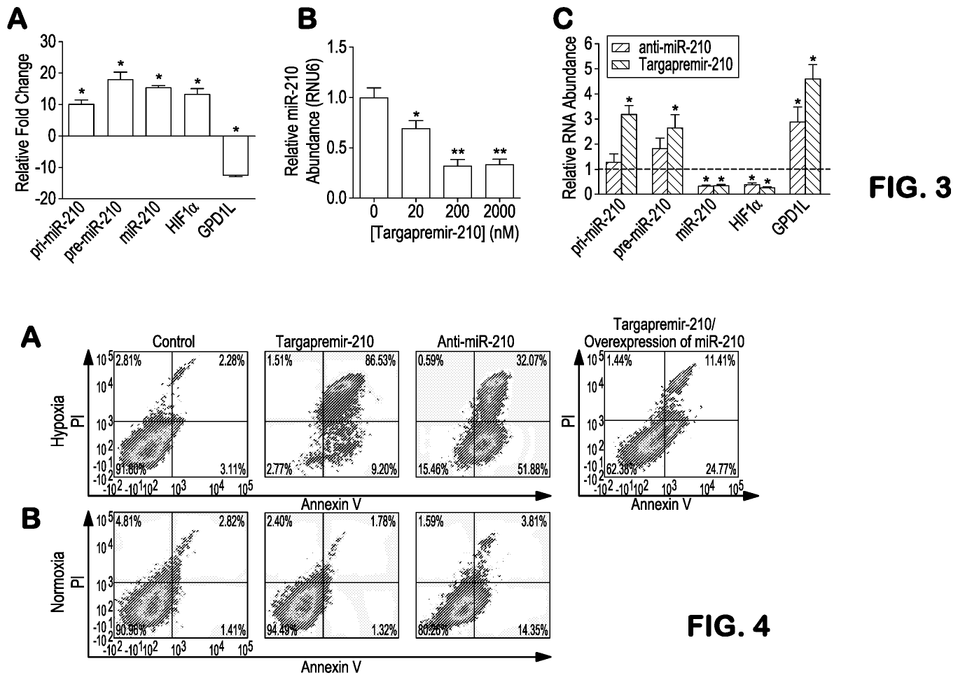

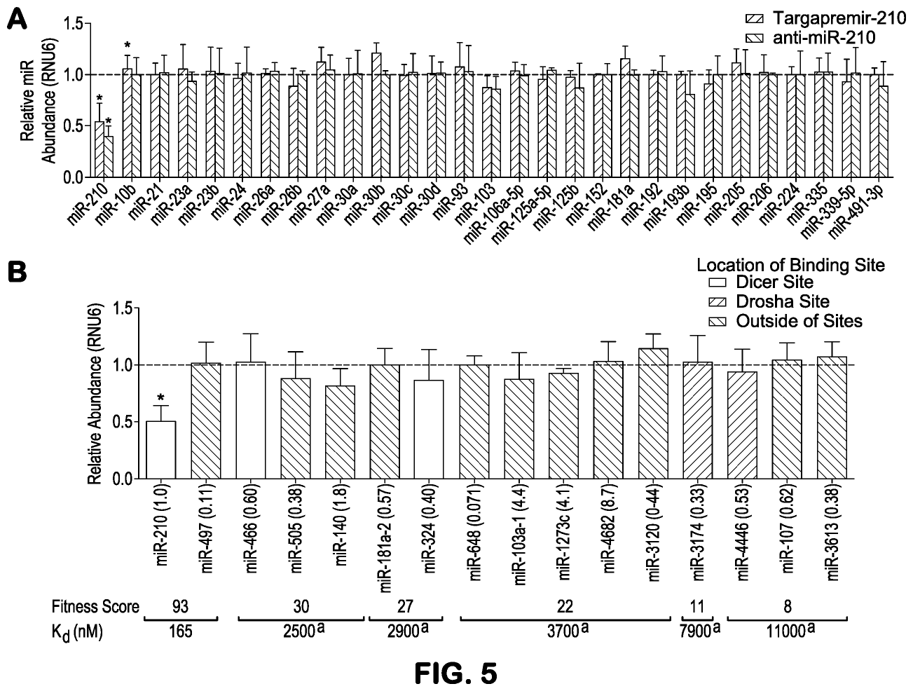

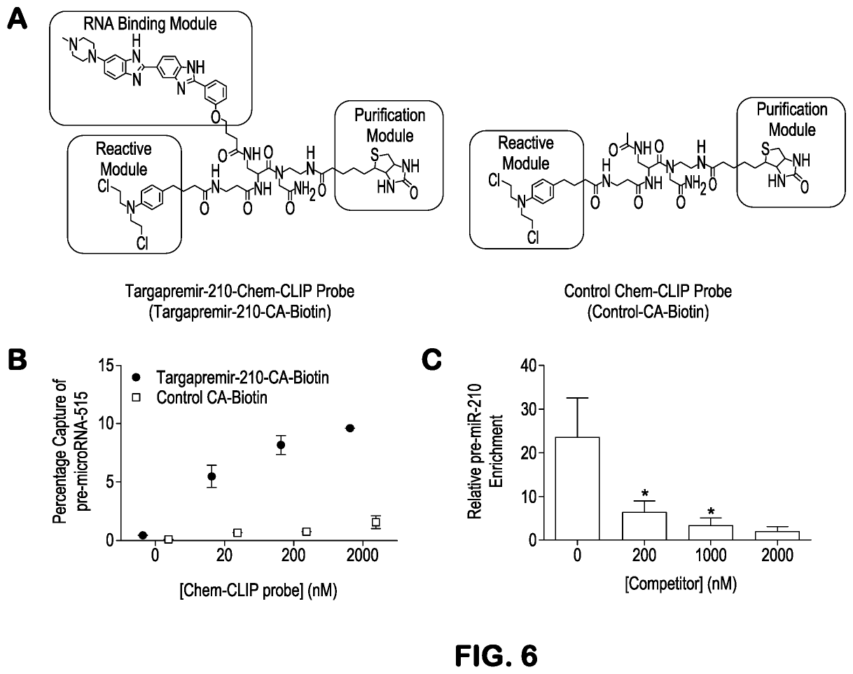

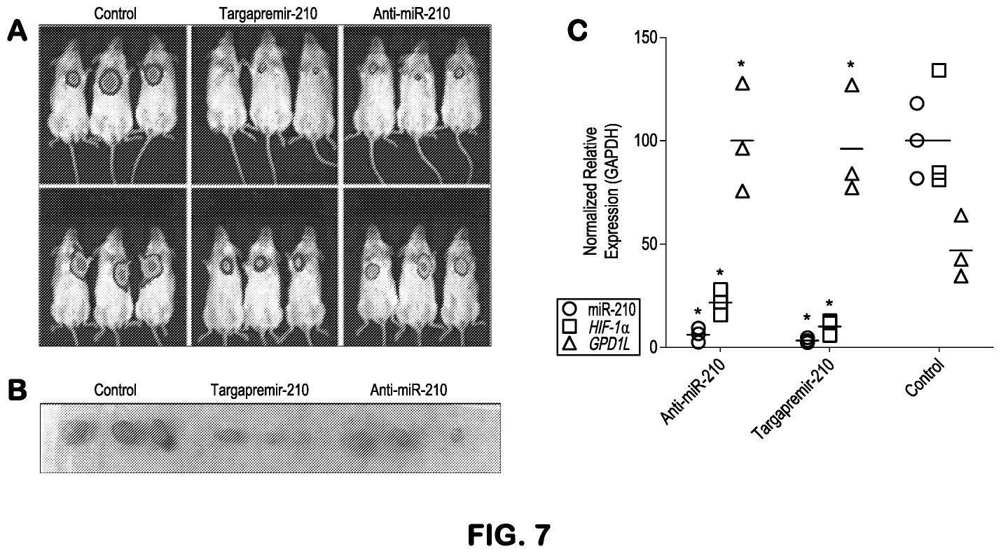

Because of the favorable functional consequences and selectivity of Targapremir-210, we completed in vivo studies of hypoxic breast cancer tumor burden. In these studies, MDA-MB-231 cells that stably express luciferase, MDA-MB-231-GFP-Luc, were generated and used for tumor implantation. MDA-MB-231-GFP-Luc cells were pre-treated with Targapremir-210 (200 nM) or anti-miR-210 antagomir (500 nM) and then implanted into mammary fat pads. Alternatively, MDA-MB-231-GFP-Luc were implanted into fat pads and mice were treated with Targapremir-210 (200 nM) or anti-miR-210 antagomir (500 nM) 24 h later with a single injection. After 21 days, tumor burden was assessed by live bioluminescent imaging. Both anti-miR-210 and Targapremir-210 significantly decreased tumor growth as assessed by luciferase signal intensity (FIG. 7A) and mass of the resected tumor (FIG. 7B). Fluorescent microscopy was used to visualize compound localization and showed that a single i.p. injection of Targapremir-210 was able to reach the tumor and sustain for the entire 21-day period.

The resected tumors were assessed for perturbation in the hypoxic signaling pathway upon compound treatment by RT-qPCR. Tumors treated with both the antagomir and Targapremir-210 expressed significantly lower levels of miR-210 and HIF-1α mRNA and significantly higher levels of GPD1L mRNA as compared to untreated tumors (FIG. 7C). Specifically, Targapremir-210 treatment reduced miR-210 and HIF-1α levels by ˜90% and ˜75%, respectively, compared to untreated tumors, while GPD1L levels were doubled. These results demonstrate that Targapremir-210 modulated its intended target (miR-210) in vivo and disrupted adaptive responses to hypoxia that promote tumor growth.

### Conclusions and Implications

In summary, these studies showed that small molecules can have cell type-specific effects by targeting non-coding RNAs and can also selectively target RNAs based on differential environmental conditions. The bioactivity of Targapremir-210 in TNBC cells and in a mouse tumor model demonstrated how RNA-binding small molecules can effectively probe a complex hypoxic circuit and potentially be developed into cancer therapeutics. Importantly, studies with the Targapremir-210 Chem-CLIP probe determined that RNA-binding small molecules can engage the desired RNA target even amongst other abundant transcripts. Furthermore, an emerging encyclopedia of information suggests that RNAs that fold into defined secondary structures but have limited tertiary structure can indeed be targeted with small molecules^{4,35,36}.

These studies validate that small molecules targeting RNA structure is a viable strategy to modulate the dysfunction of disease-associated RNAs. They also elucidate that small molecules must target a functional site to obtain a bioactive interaction for RNA. Most importantly, expression levels of different RNAs are a driver of ligand occupancy in cells. RNAs with higher expression levels are more likely to be occupied by a small molecule. Taken together, RNA may be more druggable with small molecules than anticipated.

### EXAMPLES

### Experimental Methods

### PCR Amplification & In Vitro Transcription

The DNA template for the miR-210 precursor (5′-AGCCCCTGCCCACCGCACACTGCGCTGCCCCAGACCCACTGTGCGTGTGA-CAGCGGCTGA) (SEQ ID NO: 1) was purchased from Eurofins MWG Operon and used without further purification. This template was PCR amplified in 1× PCR Buffer (10 mM Tris, pH 9.0, 50 mM KCl, and 0.1% (v/v) Triton X-100), 2 μM forward primer (5′-TAAT-ACGACTCACTATAGGAGCCCCTGCCCACCGCACAC) (SEQ ID NO: 2), 2 μM reverse primer (5′-TC-AGCCGCTGTCACACGCACA) (SEQ ID NO: 3), 4.25 mM MgCl₂, 330 μM dNTPs, and 1 μL of Taq DNA polymerase in a 600 μL reaction. PCR cycling conditions were 95° C. for 30 s, 50° C. for 30 s, and 72° C. for 60 s.

RNA was in vitro transcribed by T7 RNA polymerase in 1× Transcription Buffer (40 mM Tris-HCl, pH 8.1, 1 mM spermidine, 0.001% (v/v) Triton X-100 and 10 mM DTT) with 2.25 mM of each rNTP and 5 mM MgCl₂ at 37° C. for 18 h. The RNA was then purified on a denaturing 15% polyacrylamide gel and isolated as previously described⁴. RNA concentration was determined by UV absorbance at 260 nm at 90° C. using a Beckman Coulter DU800 UV-Vis spectrophotometer with a Peltier temperature controlling unit. Extinction coefficients were calculated using the Oligo Extinction Coefficient Calculator (https://www.scripps.edu/california/research/dna-protein-research/forms/biopolymercalc2.html).

### In Vitro Dicer Protection Assay

The miR-210 precursor was 5′-end labeled with [γ-³²P] ATP and T4 polynucleotide kinase as previously described⁴. The RNA was then folded in in 1× Reaction Buffer (Genlantis) by heating at 60° C. for 5 min and slowly cooling to room temperature, where it was then supplemented with 1 mM ATP and 2.5 mM MgCl₂. Targapremir-210 was added to the reaction mixture and the samples were allowed to incubate at room temperature for 15 min. Recombinant human Dicer enzyme (Genlantis) was added to a final concentration of 0.01 U/μL and the samples were incubated for an additional 30 min at 37 ° C. Reactions were stopped by adding in 2× Gel Loading Buffer (8 M urea, 50 mM EDTA, 0.05% (w/v) bromophenol blue, 0.05% (w/v) xylene cyanol). To generate sequencing markers, pre-miR-210 was digested with RNase T1 (0.125 U/μL) in T1 Buffer (25 mM sodium citrate, pH 5, 7 M urea, and 1 mM EDTA) for 20 min at room temperature. An RNA hydrolysis ladder was prepared by incubating RNA in 1× RNA Hydrolysis Buffer (50 mM NaHCO₃, 1 mM EDTA, pH 9.4) at 95° C. for 5 min. Cleavage products were resolved on a denaturing 15% polyacrylamide gel, which was imaged using a Molecular Dynamics Typhoon phosphorimager and quantified with Bio-Rad's QuantityOne software.

### Cell culture

MDA-MB-231 cells were cultured in RPMI 1640 medium supplemented with 10% FBS (Sigma), 1× Glutagro (Corning), and 1× Pen/Strep (MP Biomedicals, LLC) (growth medium). Cells cultured in normoxia were maintained at 37° C. in ambient atmosphere (˜21% O₂) with 5% CO₂. Cells cultured in hypoxia were maintained at 37° C., <1% O₂ in a nitrogen filled hypoxic chamber (Billups-Rothenberg, Inc.), and 5% CO₂.

### RNA isolation and RT-qPCR

Total RNA was extracted from cells using a Quick-RNA MiniPrep (Zymo Research) per the manufacturer's protocol. Approximately 200-600 ng of RNA was used in subsequent reverse transcription reactions using a miScript II RT Kit (Qiagen) or a qScript cDNA Synthesis Kit (Quanta Biosciences) per the manufacturers' recommended protocols. Primers for RT-qPCR were purchased from Integrated DNA Technologies (IDT) or Eurofins (Table S1) and used without further purification. RT-qPCR samples were prepared using Power SYBR Green PCR Master Mix (Applied Biosystems) and completed by using a 7900HT Fast Real Time PCR System (Applied Biosystems). Expression levels of RNA were normalized to U6 small nuclear RNA, GAPDH mRNA, or 18S rRNA.

### In Vitro Chem-CLIP

Growth medium was inactivated by heating at 95° C. for 15 min and then cooling to room temperature. Approximately 10,000 counts of miR-210 precursor 5′-end labeled with ³²P was added and folded at 60° C. for 5 min. After cooling, dilutions of Targapremir-210-CA-Biotin or Control CA-Biotin were added and incubated at 37° C. overnight. A 300 μL slurry of streptavidin-agarose beads (Sigma-Aldrich) was washed three times with 1× PBS and resuspended in 1× PBS. A 20 μL aliquot of the slurry was then added to the samples, which were incubated for 1 h at room temperature. The samples were centrifuged and the supernatant containing unbound RNA was transferred to a new tube. The beads were then washed three times with 1× PBS supplemented with 0.1% (v/v) Tween-20 and centrifuged, with each wash supernatant being added to the tube containing unbound RNA. The amounts of radioactivity in the supernatant and on the beads were quantified with a Beckman Coulter LS6500 Liquid Scintillation Counter.

### Cellular Chem-CLIP and C-Chem-CLIP

MDA-MB-231 cells were grown in growth medium as monolayers in 60 mm dishes to ˜60% confluency. For Chem-CLIP studies, cells were then treated with 200 nM of Targapremir-210-CA-Biotin or Control CA-Biotin; for C-Chem-CLIP studies, cells were treated with 200 nM of Targapremir-210-CA-Biotin and increasing concentrations of the parent Targapremir-210 compound. Cells were immediately placed under hypoxic conditions for 48 h, and total RNA was extracted using a Quick-RNA MiniPrep (Zymo Research) per the manufacturer's protocol. Approximately 10 μg of total RNA was incubated with 100 μL of streptavidin-agarose beads in in 1× PBS for 1 h at room temperature with gentle shaking. The solution was removed and the beads were washed 8 times with 300 μL of in 1× PBS. Bound RNA was released from the beads by heating in in 1× Elution Buffer (95% formamide, 10 mM EDTA, pH 8.2) at 60° C. for 20 min. The pulled-down RNA was purified using a Quick-RNA MiniPrep Kit (Zymo Research) and then used for subsequent RT and qPCR reactions as described above.

The relative fold-change in the amount of an RNA before and after pull-down was calculated by the ΔΔCt method as shown in equation 1:

Relative Fold−Change:2^{-(ΔC}^{ₜ} ^{before pull-down-Δc}^{ₜ} ^{after pull-down)} (1)

where the “ΔCₜ before pull-down” is the difference between the Cₜ values for the RNA of interest and a housekeeping gene (U6 small nuclear RNA, GAPDH mRNA, or 18S rRNA) in total RNA isolated from cells and “ΔCₜafter pull-down” is the difference between the Cₜ values the RNA of interest and the same housekeeping gene after pull-down.

### In Vitro Topoisomerase Inhibition Assay

Topoisomerase II inhibitory activity was measured using a Topoisomerase II Drug Screening Kit (TopoGEN, Inc.) per the manufacturer's protocol. Taragpremir-210 (200 nM) was added to 300 ng of DNA in 1× Complete Buffer (50 mM Tris-HCl, pH 8, 150 mM NaCl, 10 mM MgCl₂, 0.5 mM dithiothreitol, 30 μg/mL BSA, and 2 mM ATP), followed by addition of 7.5 U of Topoisomerase II enzyme. The samples were incubated for 30 min at 37° C., and the reaction was stopped by the addition of 2 □L of 10% sodium dodecyl sulfate (SDS). Proteinase K (50 μg/mL) was then added and the solution was incubated for 15 min at 37° C. Topoisomers were separated on 1% agarose gels with or without 0.5 μg/mL ethidium bromide. Note, for gels containing ethidium bromide, the 1× TAE running buffer was also supplemented with 0.5 μg/mL ethidium bromide. Gels prepared without ethidium bromide were post-stained (0.5 μg/mL ethidium bromide). Both types of gels were destained in 1× TAE for 15 min, and the DNA products were visualized using a Bio-Rad Gel Doc XR+imaging system.

### Mice

NOD/SCID mice (B6.CB17-Prkdcscid/Sz, Jackson Laboratories) were housed in the Scripps Florida vivarium. All live animal experiments were approved by the Scripps Florida Institutional Animal Care and Use Committee. Two sets of experiments were completed: treatment of MDA-MB-231-GFP-luc cells prior to implantation and treatment of mice post-tumor implantation. For pre-treatment of tumor cells, MDA-MB-231-GFP-luc cells (5×10⁶) were treated with or without Targapremir-210 (200 nM) or anti-miR-210 antagomir (500 nM) for 48 h pre-implantation. A 100 μL PBS/Matrigel (1:1; BD Biosciences) cell suspension was subcutaneously transplanted into mouse breast fat pads. Alternatively, mouse breast fat pads were injected with a 100 μL PBS/Matrigel (1:1) cell suspension (5×10⁶MDA-MB-231-GFP-luc cells) followed by intraperitoneal (i.p) injection of Targapremir-210 (100 μL of 200 nM) or anti-miR-210 antagomir (100 μL of 500 nM) 24 h post-transplantation.

Tumor growth was monitored weekly post-implantation by i.p. injection of 200 μL D-luciferin (15 mg/mL) and imaging with an IVIS 200 system. Tumors were resected 21 days post-transplantation following euthanasia. Levels of mature miR-210, HIF-1a, and GPLDL1 RNAs were measured by RT-qPCR as described above.

### Determination of Binding Affinities of Hoechst 33342 and Targapremir-210

Dissociation constants for the binding of pre-miR-210 RNA to Hoechst 33342 and Targapremir-210 were determined using an in solution, fluorescent binding assay. RNA was folded in 1× Binding Buffer (8 mM Na₂HPO₄, 190 mM NaCl, 1 mM EDTA, and 40 μg/mL BSA) at 60° C. for 5 min and then allowed to cool to room temperature for 10 min. Hoechst 33342 or Targapremir-210 was added to a final concentration of 500 nM. Next, 1:2 serial dilutions were performed in 1× Binding Buffer supplemented with 500 nM of Hoechst 33342 or Targapremir-210. Solutions were incubated for 30 min and then transferred to Corning non-binding surface 96-well black plates. Fluorescence intensity was then measured on a Bio-Tek FLX-800 plate reader. Change in fluorescence intensity was fit as a function of RNA concentration with equation 1:

I=I₀+0.5Δε([FL]₀−(([FL]₀+[RNA]₀Kₜ)²−4[FL]₀[RNA]₀)^{0.5}) (1)

where I and I₀ are the observed fluorescence intensity in the presence and absence of RNA, respectively, Δε is the difference between the fluorescence intensity in the absence and in the presence of infinite RNA concentration, [FL]₀ and [RNA]₀ are the concentrations of compound (Hoechst 33342 or Targapremir-210) and RNA, respectively, and Kₜ is the dissociation constant.

### Microarray Analysis of miRNA

MDA-MB-231 cells were treated with either a miRNA antagomir against miR-210 or Targapremir-210 and placed under hypoxic conditions for 3 days. Total RNA was isolated using the Quick-RNA MiniPrep kit (Zymo Research) and poly (A)-tailed and biotin-labeled using the FlashTag Biotin HSR RNA Labeling Kit (Affymetrix). Biotin-labeled RNA samples were hybridized to the GeneChip miRNA 4.0 microarray (Affymetrix). Following hybridization, microarrays were washed and stained using the Fluidics Station 450 (Affymetrix) and scanned using the GeneChip Scanner 3000 7G (Affymetrix). Expression analysis was carried out using the Expression Console Software package (Affymetrix).

### Tumor Localization of Targapremir-210

Resected tumor samples were frozen using O.C.T. Compound (Tissue-Tek) over dry ice. Frozen tissues were sectioned by cryostat and transferred to microscope slides. Fluorescent microscopic analysis was carried out using a Leica DMI3000 B upright fluorescent microscope.

### SYNTHETIC METHODS

Abbreviations: Boc, tert-butyloxycarbonyl; DCM, dichloromethane; DIC, N,N′-Diisopropylcarbodiimide; DIEA, N,N-Diisopropylethylamine; DMF, N,N-dimethylformamide; Fmoc, fluorenylmethoxycarbonyl chloride; HATU, 1-[Bis(dimethylamino)methylene-]1H-1,2,3-trizaolo[4,5-b]pyridinium 3-oxide hexafluorophosphate; HOAt, 1-hydroxy-7-azabenzotriazole; HPLC, high performance liquid chromatography; MALDI-TOF, matrix-assisted laser desorption/ionization-time of flight; TFA, trifluoracetic acid

### Targapremir-210

Targapremir-210 was synthesized as previously described.¹

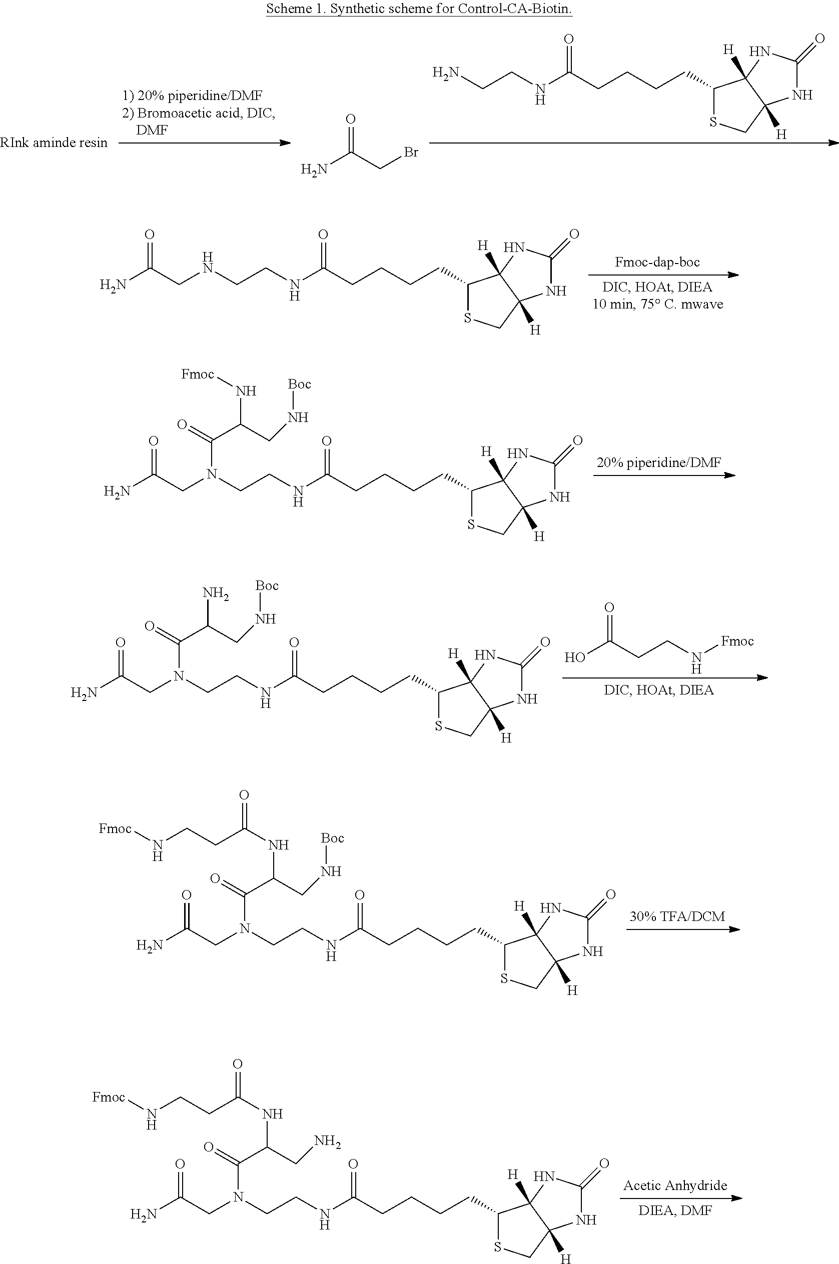

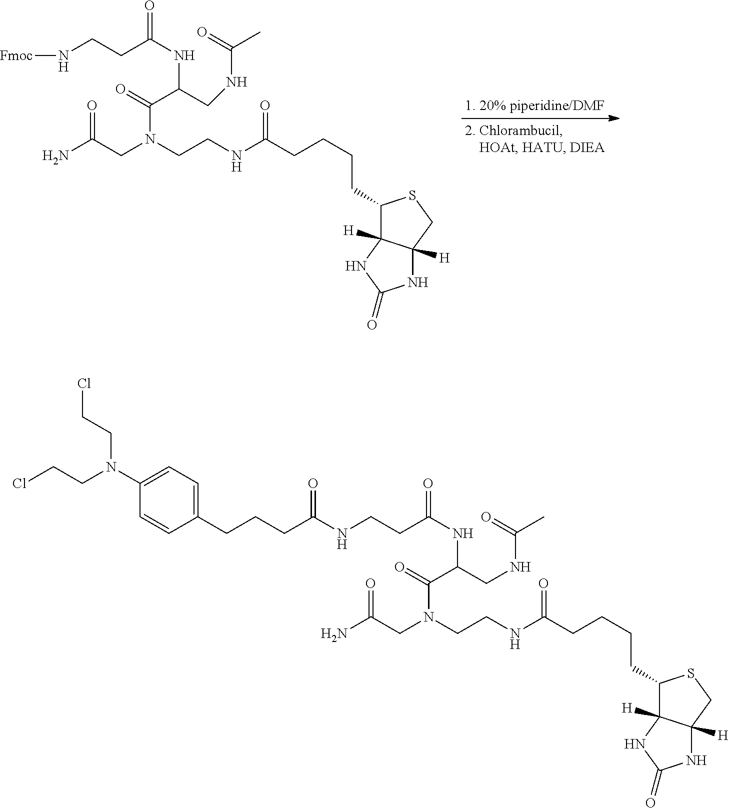

### Synthesis of Control-CA-Biotin (Control Chem-CLIP Probe Lacking the RNA-Binding Module)

Rink amide resin (1 g, 0.6 mmol) was swollen in DCM for 5 min and then in DMF for 5 min. The resin was de-protected with a solution of 20% piperidine in DMF (5 mL, 2×20 min). After washing with DMF (3×5 mL), the resin was treated with a solution of bromoacetic acid (0.412 g, 3 mmol) and DIC (0.464 mL, 3 mmol) in DMF (5 mL) via microwave irradiation (3×15 s) using a 700 W microwave set to 10% power. The resin was washed with DMF (3×5 mL) and then reacted with a solution of N-(4-aminoethyl)-biotin (344 mg, 1.2 mmol) and DIEA (0.3 mL, 0.6 mmol) in DMF (4 mL) via microwave irradiation (3×15 s) using a 700 W microwave set to 10% power. The resin was washed with DMF (3×5 mL) and treated with a solution of Fmoc-Dap(Boc)-OH (0.768 g, 1.8 mmol), HOAt (0.25 g, 1.8 mmol), and DIC (0.464 mL, 3 mmol) via microwave irradiation (3×15 s) using a 700 W microwave set to 10% power. The resin was treated with a solution of 20% piperidine in DMF (4 mL), washed with DMF, and then treated with a solution of Fmoc-β-alanine (0.56 g, 1.8 mmol), HOAt (0.25 g, 1.8 mmol), DIC (0.464 mL, 3 mmol), and DIEA (0.3 mL, 1.8 mmol) in DMF (3 mL) via microwave irradiation (3×15 s) using a 700 W microwave set to 10% power. The Boc protecting group was removed with a solution of 30% TFA in DCM (4 mL) for 15 min at room temperature. The solution was concentrated in vacuo and azeotroped with toluene three times. The resulting oil was purified by preparative HPLC using a linear gradient of 20-100% CH₃OH in H₂O with 0.1% TFA over 60 min. The purified compound (0.036 g, 0.05 mmol) was treated with a solution of acetic anhydride (0.05 mL, 0.53 mmol) and DIEA (0.1 mL, 0.6 mmol) in DMF (4 mL) via microwave irradiation (3 ×15 s) using a 700 W microwave set to 10% power. The reaction was concentrated under vacuum and purified by preparative HPLC using a linear gradient of 20-100% CH₃OH in H₂O with 0.1% TFA over 60 min. Fractions containing the product were dissolved in 20% piperidine in DMF (2 mL) and stirred for 20 min. The reaction mixture was concentrated under vacuum and purified by preparative HPLC using a linear gradient of 20-100% CH₃OH in H₂O with 0.1% TFA over 60 min.

The resulting purified compound was treated with a solution of chlorambucil (5 mg, 0.01 mmol), HOAt (2.72 mg, 0.02 mmol), HATU (7.6 mg, 0.02 mmol) and DIEA (0.016 mL, 006 mmol) at room temperature for 3 h. The mixture was then concentrated under vacuum and purified via preparative HPLC using a linear gradient of 0-100% CH₃CN in H₂O with 0.1% TFA over 60 min. Isolated 533 nmol; 0.06% yield. Retention time: 38 min; hydrolyzed product retention time: 39 min. HRMS calculated: 828.3395 (M+H), mass found: 828.4584 (M+H).

Hydrolyzed product, HRMS calculated: 792.4078 (M+H), mass found: 792.4839 (M+H).
Compound can hydrolyze during purification due to reactive alkyl halogen groups.

### Documents Cited

(1) Gerstein, M. B.; Bruce, C.; Rozowsky, J. S.; Zheng, D.; Du, J.; Korbel, J. O; Emanuelsson, O.; Zhang, Z. D.; Weissman, S.; Snyder, M. Genome Res. 2007, 17, 669.
(2) Clamp, M.; Fry, B.; Kamal, M.; Xie, X.; Cuff, J.; Lin, M. F.; Kellis, M.; Lindblad-Toh, K.; Lander, E. S. Proc. Natl. Acad. Sci. U.S.A. 2007, 104, 19428.
(3) Childs-Disney, J. L.; Wu, M.; Pushechnikov, A.; Aminova, O.; Disney, M. D. ACS Chem. Biol. 2007, 2, 745.
(4) Velagapudi, S. P.; Gallo, S. M.; Disney, M. D. Nat. Chem. Biol. 2014, 10, 291.
(5) Disney, M. D.; Winkelsas, A. M.; Velagapudi, S. P.; Southern, M.; Fallahi, M.; Childs-Disney, J. L. ACS Chem. Biol. 2016, 11, 1720.
(6) He, L.; Hannon, G. J. Nat. Rev. Genet .2004, 5, 522.
(7) Bartel, D. P. Cell 2004, 116, 281.
(8) Behm-Ansmant, I.; Rehwinkel, J.; Izaurralde, E. Cold Spring Harb. Symp. Quant. Biol. 2006, 71, 523.
(9) Bartel, D. P. Cell 2009, 136, 215.
(10) Kelly, T. J.; Souza, A. L.; Clish, C. B.; Puigserver, P. Mol. Cell. Biol. 2011, 31, 2696.
(11) Redova, M.; Poprach, A.; Besse, A.; Illiev, R.; Nekvindova, J.; Lakomy, R.; Radova, L.; Svoboda, M.; Dolezel, J.; Vyzula, R.; Slaby, O. Tumour Biol .2013, 34, 481.
(12) Grosso, S.; Doyen, J.; Parks, S. K.; Bertero, T.; Paye, A.; Cardinaud, B.; Gounon, P.; Lacas-Gervais, S.; Noel, A.; Pouyssegur, J.; Barbry, P.; Mazure, N. M.; Mari, B. Cell Death Dis. 2013, 4, e544.
(13) Maxwell, P. H.; Wiesener, M. S.; Chang, G. W.; Clifford, S. C.; Vaux, E. C.; Cockman, M. E.; Wykoff, C. C.; Pugh, C. W.; Maher, E. R.; Ratcliffe, P. J. Nature 1999, 399, 271.
(14) Semenza, G. L. Nat Rev Cancer 2003, 3, 721.
(15) Velagapudi, S. P.; Seedhouse, S. J.; French, J.; Disney, M. D. J. Am. Chem. Soc. 2011, 133, 10111.
(16) Rothé, F.; Ignatiadis, M.; Chaboteaux, C.; Haibe-Kains, B.; Kheddoumi, N.; Majjaj, S.; Badran, B.; Fayyad-Kazan, H.; Desmedt, C.; Harris, A. L.; Piccart, M.; Sotiriou, C. PLoS One 2011, 6, e20980.
(17) Blancher, C.; Moore, J. W.; Talks, K. L.; Houlbrook, S.; Harris, A. L. Cancer Res. 2000, 60, 7106.
(18) Greijer, A. E.; van der Wall, E. J. Clin. Pathol. 2004, 57, 1009.
(19) Kilic, M.; Kasperczyk, H.; Fulda, S.; Debatin, K. M. Oncogene 2006, 26, 2027.
(20) Kulshreshtha, R.; Ferracin, M.; Wojcik, S. E.; Garzon, R.; Alder, H.; Agosto-Perez, F. J.; Davuluri, R.; Liu, C.-G.; Croce, C. M.; Negrini, M.; Calin, G. A.; Ivan, M. Mol. Cell. Biol. 2007, 27, 1859.
(21) Alterio, V.; Di Fiore, A.; D'Ambrosio, K.; Supuran, C. T.; De Simone, G. Chem. Rev. 2012, 112, 4421.
(22) Thorpe, L. M.; Yuzugullu, H.; Zhao, J. J. Nat. Rev. Cancer 2015, 15, 7.
(23) Liu, B.; Childs-Disney, J. L.; Znosko, B. M.; Wang, D.; Fallahi, M.; Gallo, S. M.; Disney, M. D. BMC Bioinformatics 2016, 17, 1.
(24) Guan, L.; Disney, M. D. Angew. Chem. Int. Ed. Engl. 2013, 52, 10.1002/anie.201301639.
(25) Yang, W.-Y.; Gao, R.; Southern, M.; Sarkar, P. S.; Disney, M. D. Nat. Commun. 2016, 7.
(26) Mathews, D. H.; Disney, M. D.; Childs, J. L.; Schroeder, S. J.; Zuker, M.; Turner, D. H. Proc. Natl. Acad. Sci. U.S.A. 2004, 101, 7287.
(27) Wells, S. E.; Hughes, J. M.; Igel, A. H.; Ares, M., Jr. Methods Enzymol. 2000, 318, 479.
(28) Kwok, C. K.; Ding, Y.; Tang, Y.; Assmann, S. M.; Bevilacqua, P. C. Nat. Commun. 2013, 4, 2971.
(29) Spitale, R. C.; Crisalli, P.; Flynn, R. A.; Torre, E. A.; Kool, E. T.; Chang, H. Y. Nat. Chem. Biol. 2013, 9, 18.
(30) Deigan, K. E.; Li, T. W.; Mathews, D. H.; Weeks, K. M. Proc. Natl. Acad. Sci. U.S.A. 2009, 106, 97.
(31) Lee, M. M.; Childs-Disney, J. L.; Pushechnikov, A.; French, J. M.; Sobczak, K.; Thornton, C. A.; Disney, M. D. J. Am. Chem. Soc. 2009, 131, 17464.
(32) Childs-Disney, J. L.; Tsitovich, P. B.; Disney, M. D. Chembiochem 2011, 12, 2143.
(33) Beerman, T. A.; McHugh, M. M.; Sigmund, R.; Lown, J. W.; Rao, K. E.; Bathini, Y. Biochim. Biophys. Acta Gene Struct. Exp. 1992, 1131, 53.
(34) Nitiss, J. L. Nat Rev Cancer 2009, 9, 338.
(35) Velagapudi, S. P.; Cameron, M. D.; Haga, C. L.; Rosenberg, L. H.; Lafitte, M.; Duckett, D. R.; Phinney, D. G.; Disney, M. D. Proc. Natl. Acad. Sci. U.S.A. 2016, 113, 5898.
(36) Kumar, A.; Parkesh, R.; Sznajder, L. J.; Childs-Disney, J. L.; Sobczak, K.; Disney, M. D. ACS Chem. Biol. 2012, 7, 496.

What is claimed is:

1. A method of inhibiting biogenesis of microRNA miR-210 in a cell under hypoxic conditions, comprising contacting the cell with an effective amount or concentration of a compound of formula

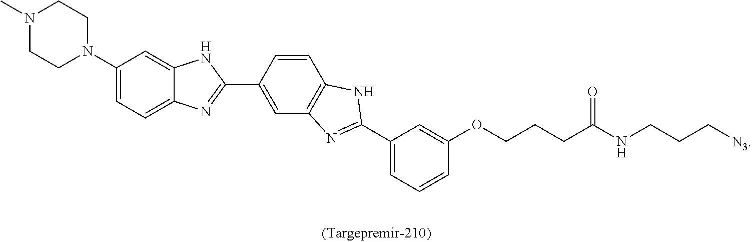 [SMILES: CN1CCN(c2ccc3nc(-c4ccc5[nH]c(-c6cccc(OCCCC(=O)NCCCN=[N+]=[N-])c6)nc5c4)[nH]c3c2)CC1]

2. The method of claim 1, wherein the Targapremir-210 selectively recognizes the miR-210 precursor and can differentially recognize RNAs in cells that have the same target motif but have different expression levels.

3. A method of target profiling a set of cells for expression of a miR-210 microRNA precursor, comprising administering an effective amount or concentration of Targapermir-210 to the set of cells, then screening the set of cells for expression or suppression of a phenotype associated with miR-210.

4. A method of impeding tumor proliferation in a mammal in vivo, comprising administering to the animal an effective dose of a compound of of formula

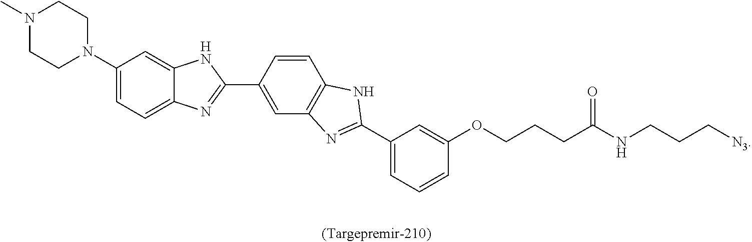 [SMILES: CN1CCN(c2ccc3nc(-c4ccc5[nH]c(-c6cccc(OCCCC(=O)NCCCN=[N+]=[N-])c6)nc5c4)[nH]c3c2)CC1]

5. The method of claim 4, wherein the tumor is breast cancer.
